(19) United States
(12) Patent Application Publication
(10) Pub. No.: US 2008/0299072 A1
(43) Pub. Date: Dec. 4, 2008

(54) Chemokine Antagonists

(75) Inventors: Amanda Proudfoot, Chens Sur Leman (FR); Maud Deruaz, Carouge (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: LABORATOIRES SERONO SA, Coinsins, Vaud (CH)

(21) Appl. No.: 12/094,905

(22) PCT Filed: Dec. 14, 2006
(86) PCT No.: PCT/EP2006/069709

§ 371 Date: May 23, 2008

Related U.S. Application Data
(60) Provisional application No. 60/751,615, filed on Dec. 19, 2005.

(30) Foreign Application Priority Data
Dec. 15, 2005 (EP) 05112223.2

(51) Int. Cl. A61K 38/19; C07K 14/52; A61P 37/00; A61P 29/00; A61P 31/00

(52) U.S. Cl. 424/85.1; 530/350

(57) ABSTRACT

New CC-chemokine antagonists are provided. Compounds prepared in accordance with the present invention can be used as anti-inflammatory and immunomodulatory compounds and in the treatment or prevention of CC-chemokine-related diseases.

### FIELD OF THE INVENTION

The invention relates to novel amino-terminally PEGylated CC-chemokines acting as CC-chemokine antagonists.

### BACKGROUND OF THE INVENTION

Chemokines are secreted pro-inflammatory proteins of small dimensions (70-130 amino acids) mostly involved in the directional migration and activation of cells, especially the extravasation of leukocytes from the blood to tissue localizations needing the recruitment of these cells. Usually chemokines are produced at the site of an injury, inflammation, or other tissue alteration in a paracrine or autocrine fashion, triggering cell-type specific migration and activation.

Depending on the number and the position of the conserved cysteines in the sequence, chemokines are classified into C, CC, CXC and CX₃C chemokines. Inside each of these families, chemokines can be further grouped according to the homology of the entire sequence, or of specific segments.

A series of heptahelical G-protein coupled membrane receptors, are the binding partners that allow chemokines to exert their biological activity on the target cells, which present specific combinations of receptors according to their state and/or type. An unified nomenclature for chemokine ligands and receptors, which were originally named by the scientists discovering them in a very heterogeneous manner, has been proposed to associate each of these molecules to a systematic name including a progressive number: CCL1 CCL2, etc. for CC chemokines; CCR1CCR2, etc. for CC chemokines receptors, and so on.

The physiological effects of chemokines result from a complex and integrated system of concurrent interactions. The receptors often have overlapping ligand specificity, so that a single receptor can bind different chemokines, as well as a single chemokine can bind different receptors. In particular, the N-terminal domain of chemokines is involved in receptor binding and N-terminal processing can either activate chemokines or render chemokines completely inactive.

These molecules offer the possibility for therapeutic intervention in pathological conditions associated to such processes, in particular by inhibiting/antagonizing specific chemokines and their receptors at the scope to preventing the excessive recruitment and activation of cells, in particular leukocytes, for a variety of indications related to inflammatory and autoimmune diseases, cancers, and bacterial or viral infections (Proudfoot A et al., 2000). Amongst all the chemokines characterized so far, CC-chemokines, such as CCL5 (also known as RANTES; Appay V and Rowland-Jones S L, 2001) have been intensively studied to identify therapeutically useful molecules. Variants of CC-chemokines, missing up to nine N-terminal amino acids, have been tested for their activity as inhibitors or antagonists of the naturally occurring forms. These molecules are inactive on monocytes and are useful as receptor antagonists (Gong J and Clark-Lewis I, 1995; Gong J H et al., 1996; WO 99/16877). Alternatively, N-terminal extension of the mature CC-chemokine with one Methionine results in almost complete inactivation of the molecule, which also behaves as an antagonist for the authentic one (WO 96/17935).

Moreover, in order to perform structure-function analysis of CC-chemokines, variants containing substitutions or chemical modifications in different positions, as well as CC-chemokine derived peptides, have been tested for the interactions with receptors or other molecules, such as Glycosaminoglycans (GAGs). Some of these variants have been disclosed as having significantly altered binding properties, and sometimes they are active as CC-chemokine antagonists, having potential therapeutic applications in the treatment of HIV infection and inflammatory or allergic diseases (WO 99/33989; WO 02/28419).

However, there is a need for novel and improved CC-chemokine antagonists that may be used in the treatment of inflammatory and autoimmune diseases.

### SUMMARY OF THE INVENTION

It has been surprisingly found that amino-terminally PEGylated CC chemokines such as RANTES, wherein the poly(ethyleneglycol) is attached to the amino-terminal residue via the α-amino group, have antagonistic activity.

They can be used in the treatment of inflammatory and autoimmune diseases, cancers, and bacterial or viral infections.

Other features and advantages of the invention will be apparent from the following detailed description.

### DESCRIPTION OF THE FIGURES

FIG. 1: Equilibrium competition binding to CCR1. The binding assay was performed by monitoring the displacement of [¹²⁵I]-MIP-1α from CCR1-expressing CHO membranes. A) PEGylated Ser/Cys variants (S1C, S4C). Added as competitors were: RANTES (□), Met-RANTES (X), S1C-5 kDaPEG-RANTES (▪), S4C-5 kDaPEG-RANTES (). B) N-terminal PEGylated wt RANTES variants. Added as competitors were: RANTES (□), Met-RANTES (X), PEG-2 kDa-RANTES (▴), PEG-5 kDa-RANTES (▾). The IC₅₀ values are 69 nM for RANTES, 72 nM for Met-RANTES, 272 nM for S1C-5 kDaPeg-RANTES, 83 nM for S4C-5 kDaPeg-RANTES, 41 nM for Peg-2 kDa-RANTES and 47 nM for Peg-5 kDa-RANTES.

FIG. 2: Equilibrium competition binding to CCR5. The binding assay was performed by monitoring the displacement of [¹²⁵I]-MIP-1α from CCR5-expressing CHO membranes. A) PEGylated Ser/Cys variants. Added as competitors were: RANTES (□), Met-RANTES (x), S1C-5 kDaPEG-RANTES (▪), S4C-5 kDaPEG-RANTES (), S5C-5 kDaPEG-RANTES (□). B) N-terminal PEGylated wt RANTES variants. Added as competitors were RANTES (□), Met-RANTES (x), PEG-2 kDa-RANTES (▴), PEG-5 kDa-RANTES (▾). The IC₅₀ values are 1.3 nM for RANTES, 15.5 nM for Met-RANTES, 82.5 nM for S1C-5 kDaPEG-RANTES, 45.4 nM for S4C-5 kDaPEG-RANTES, 902 nM for S5C-5 kDaPEG-RANTES, 3.1 nM for PEG-2 kDa-RANTES and 2.2 nM for PEG-5 kDa-RANTES.

FIG. 3: Ability to induce chemotaxis of L1.2/CCR5 transfectants. A graph representing the results of the transwell chemotaxis assay performed using L1.2/CCR5 transfectants and as chemotactic agent, A) RANTES (□), Met-RANTES (X), S1C-5 kDaPEG-RANTES (▪), S4C-5 kDaPEG-RANTES (). B) RANTES (□), Met-RANTES (X) PEG-5 kDa-RANTES (▾).

FIG. 4: Ability to inhibit chemotaxis of L1.2/CCR5 transfectants induced by 1 nM RANTES. A graph representing the results of the transwell chemotaxis assay performed using L1.2/CCR5 transfectants and as chemotactic agent RANTES (□) with as competitors, Met-RANTES (X), S1C-5 kDaPEG-RANTES (▪), S4C-5 kDaPEG-RANTES (). B) Met-RANTES (X), PEG-2 kDa-RANTES (▴), PEG-5 kDa-RANTES (▾).

FIG. 5: Inhibition of irritant induced contact hypersensitivity in vivo. The area under the curve shows the ear swelling of mice after irritation with 2% croton oil in acetone/olive oil at time 0 and treated after 30′ with RANTES antagonist, either Met-RANTES or Peg-2 kDa-RANTES or Peg-5 kDa-RANTES at 0.5 mg/kg via i.p. injection or Dexamethasone at 0.5 mg/kg via s.c. as control.

### DETAILED DESCRIPTION OF THE INVENTION

On the basis of equilibrium competition binding and chemotaxis assays, we have now found that amino-terminally PEGylated RANTES (also referred to as CCL5), in which poly(ethyleneglycol) is attached to the amino-terminal residue via the α-amino group, acts as RANTES antagonist, useful in the treatment of inflammatory and autoimmune diseases, cancers, and bacterial or viral infections. Amino-terminally PEGylated CC-chemokines, in which poly(ethyleneglycol) is attached to the amino-terminal residue via the α-amino group, in general have antagonistic activity. This is supported by structure and sequence similarity of RANTES with other known CC-chemokines.

Prior art fails to teach any amino-terminally PEGylated chemokines.

Therefore the main object of the present invention is to provide amino-terminally PEGylated CC-chemokines wherein poly(ethyleneglycol) is attached to the amino-terminal residue via the α-amino group. Human CC-chemokines sharing consensus sequences with RANTES, such as CCL1, CCL2, CCL3, CCL4, CCL5, CCL7, CCL11, CCL13 and CCL15 are preferred. RANTES and CCL-2 (MCP-1) are more preferred. In a particularly preferred embodiment, the amino-terminally PEGylated CC-chemokine is human wildtype RANTES having the amino acid sequence of SEQ ID NO:1.

Preferably, the amino-terminally PEGylated CC-chemokines according to the invention have reduced receptor activation ability compared to their respective unpegylated wildtype CC-chemokines.

Preferably, the amino-terminally PEGylated CC-chemokines according to the invention are chemotaxis inhibitors.

Preferably, the amino-terminally PEGylated CC-chemokines according to the invention antagonize the activity of their respective unpegylated wildtype CC-chemokines.

The examples show that RANTES, which is PEGylated on amino acid side chains instead of the α-amino group of the amino terminal residue, has no antagonistic activity. This is true even if the PEGylation takes place on the side chain of the amino-terminal amino acid.

The chemokines of the present invention can be provided in alternative forms which can be preferred according to the desired method of use and/or production, for example as active fractions, precursors, salts, derivatives, conjugates or complexes.

The “precursors” are compounds, which can be converted into the compounds of the present invention by metabolic, or enzymatic processing prior to or after the administration to the cells or to the organism.

The term “salts” herein refers to both salts of carboxyl groups and to acid addition salts of amino groups of the peptides, polypeptides, or analogs thereof, of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the C-terminal groups according to known methods. Such derivatives include for example esters or aliphatic amides of the carboxyl-groups and N-acyl derivatives of free amino groups or O-acyl derivatives of free hydroxyl-groups and are formed with acyl-groups as for example alcanoyl- or aroyl-groups. Alternatively, the derivatives may contain sugars or phosphates groups linked to the functional groups present on the lateral chains of the amino acid moieties. Such molecules can result from in vivo or in vitro processes which do not normally alter primary sequence, for example chemical derivativization of peptides (acetylation or carboxylation), phosphorylation (introduction of phosphotyrosine, phosphoserine, or phosphothreonine residues) or glycosylation (by exposing the peptide to enzymes which affect glycosylation e.g., mammalian glycosylating or deglycosylating enzymes).

Useful conjugates or complexes of the antagonists of the present invention can be generated, using molecules and methods known in the art for improving the detection of the interaction with other proteins (radioactive or fluorescent labels, biotin), therapeutic efficacy (cytotoxic agents, isotopes), or drug delivery efficacy.

“N-terminal PEGylation” according to this invention means the attachment of a poly(ethyleneglycol) polymer (PEG) to the α-amino group of the amino-terminal amino acid of the CC-chemokine. The grafting of PEG chains or PEG-based chains onto proteins is known.

See for example U.S. Pat. No. 5,122,614 which describes that PEG is converted into its N-succinimide carbonate derivative. Also known are PEG chains modified with reactive groups to facilitate grafting onto proteins. See for example U.S. Pat. No. 5,739,208 which describes a PEG derivative that is activated with a sulfone moiety for selective attachment to thiol moieties on molecules and surfaces, or U.S. Pat. No. 5,672,662 which discloses active esters of PEG. Alternatively, aldehyde coupled PEG may be used. PEG may be either linear or branched but is preferably linear.

In one embodiment, the present invention also encompasses a process for the preparation of an amino-terminally PEGylated CC-chemokine wherein poly(ethyleneglycol) is attached to the amino-terminal residue via the α-amino group, characterized in that the CC-chemokine is reacted with a PEG via an aldehyde group, preferably by using mPEG-ButyrALD (mPEG-butyl-aldehyde) in the presence of BH₃CNN as catalyst. In this case PEGylation is performed by an acylation reaction.

The CC-chemokines according to the present invention can be recombinantly produced. Expression of any of the recombinant proteins of the invention as mentioned herein can be effected in Eukaryotic cells (e.g. yeasts, insect or mammalian cells) or Prokaryotic cells, using the appropriate expression vectors. Any method known in the art can be employed. In particular, mammalian cells, such as human, monkey, mouse, and Chinese hamster ovary (CHO) cells in particular, are preferred because they provide post-translational modifications to protein molecules, including correct folding or glycosylation at correct sites. Also yeast cells can carry out post-translational peptide modifications including glycosylation. A number of recombinant DNA strategies exist which utilize strong promoter sequences and high copy number of plasmids which can be utilized for production of the desired proteins in yeast. Yeast recognizes leader sequences on cloned mammalian gene products and secretes peptides bearing leader sequences (i.e., pre-peptides). Alternatively, any of the specific protocols for the expression of chemokines in bacterial cells disclosed in the literature can be used (Edgerton M D et al., 2000). Factors of importance in selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species.

The vectors should allow the expression of the isolated or fusion protein including the antagonist of the invention in the Prokaryotic or Eukaryotic host cell under the control of transcriptional initiation/termination regulatory sequences, which are chosen to be constitutively active or inducible in said cell. After the introduction of the vector(s), the host cells are grown in a selective medium, which selects for the growth of vector-containing cells. Expression of the cloned gene sequence(s) results in the production of the desired proteins. A cell line substantially enriched in such cells can be then isolated to provide a stable cell line.

For Eukaryotic hosts (e.g. yeasts, insect or mammalian cells), different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells which have been stably transformed by the introduced DNA can be selected by also introducing one or more markers which allow for selection of host cells which contain the expression vector. The marker may also provide for prototrophy to an auxotrophic host, biocide resistance, e.g. antibiotics, or heavy metals such as copper, or the like. The selectable marker gene can either be directly linked to the DNA gene sequences to be expressed, or introduced into the same cell by co-transfection.

These objects of the invention can be achieved by combining the disclosure provided by the present patent application on antagonists of CC-chemokines, with the knowledge of common molecular biology techniques.

The amino-terminally PEGylated CC-chemokines of the invention may be prepared by any other well known procedure in the art, in particular, by the well established chemical synthesis procedures, which can be efficiently applied on these molecule given the short length. Totally synthetic CC-chemokines, also containing additional chemical groups, are disclosed in the literature (Brown A et al., 1996; Vita C et al., 2002).

Examples of chemical synthesis technologies are solid phase synthesis and liquid phase synthesis. As a solid phase synthesis, for example, the amino acid corresponding to the carboxy-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc (2,2,5,7,8-pentamethylchromane-6-sulphonyl) for the guanidino groups; and tBu (t-butyl) for the hydroxyl groups. After synthesis of the desired peptide, it is subjected to the de-protection reaction and cut out from the solid support. Such peptide cutting reaction may be carried out with hydrogen fluoride or tri-fluoromethane sulfonic acid for the Boc method, and with TFA for the Fmoc method. Finally, the intact full-length peptides are purified and chemically or enzymatically folded (including the formation of disulphide bridges between cysteines) into the corresponding CC-chemokines of the invention.

Purification of the natural, synthetic or recombinant proteins is carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies, heparin, or any other suitable ligand which can bind the target protein at high efficiency and can be immobilized on a gel matrix contained within a column. Impure preparations containing the proteins are passed through the column. The protein will be bound to the column by means of this ligand while the impurities will pass through. After washing, the protein is eluted from the gel by a change in pH or ionic strength. Alternatively, HPLC (High Performance Liquid Chromatography) can be also used.

Another object of the present invention is the use of the amino-terminally PEGylated CC-chemokine as above defined as medicaments, in particular as the active ingredients in pharmaceutical compositions (and formulated in combination with pharmaceutically acceptable carriers, excipients, stabilizers, adjuvants, or diluents). Still another object is the use of the amino-terminally PEGylated CC-chemokine of the invention to produce a pharmaceutical composition for treating or preventing disorders in which the antagonistic properties of said molecules can provide beneficial effects such as, according to the literature on chemokines, autoimmune and inflammatory diseases, cancers, as well as bacterial and viral infections. A non-limitative list of specific disorders includes arthritis, rheumatoid arthritis (RA), psoriatic arthritis, osteoarthritis, systemic lupus erythematosus (SLE), systemic sclerosis, scleroderma, polymyositis, glomerulonephritis, melanoma, carcinoma, leukaemia, lymphoblastoma, liver fibrosis, skin fibrosis, lung fibrosis, allergic or hypersensitivity diseases, dermatitis, Type IV hypersensitivity also called delayed-type hypersensitivity or DTH, asthma, chronic obstructive pulmonary disease (COPD), inflammatory bowel disease (IBD), Crohn's diseases, ulcerative colitis, multiple sclerosis, septic shock, HIV-infection, transplantation, graft-versus-host disease (GVHD), atherosclerosis. Preferably the amino-terminally PEGylated CC-chemokine of the invention is used in the treatment or prevention of inflammatory diseases, more preferably atopical diseases and particularly preferably contact dermatitis.

Another object of the present invention is, therefore, the method for treating or preventing any of the above mentioned diseases by administering an effective amount of the amino-terminally PEGylated CC-chemokine of the invention together with a pharmaceutically acceptable excipient, and/or with another therapeutic composition which acts synergistically or in a coordinated manner with the amino-terminally PEGylated CC-chemokine of the invention. For example, synergistic properties of CC-chemokine antagonists have been demonstrated in combination with cyclosporin (WO 00/16796).

An “effective amount” refers to an amount of the active ingredients that is sufficient to affect the course and the severity of the disease, leading to the reduction or remission of such pathology. The effective amount will depend on the route of administration and the condition of the patient.

A further object of the present invention are the pharmaceutical compositions containing the amino-terminally PEGylated CC-chemokine of the invention, in the presence of one or more pharmaceutically acceptable carriers, for treating or preventing any of the above mentioned diseases. The pharmaceutical compositions may be formulated in any acceptable way to meet the needs of the mode of administration. For example, the use of biomaterials and other polymers for drug delivery, as well the different techniques and models to validate a specific mode of administration, are disclosed in literature (Luo B and Prestwich G D, 2001; Cleland J L et al., 2001).

“Pharmaceutically acceptable” is meant to encompass any carrier, which does not interfere with the effectiveness of the biological activity of the active ingredient and that is not toxic to the host to which is administered. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil). For example, for parenteral administration, the above active ingredients may be formulated in unit dosage form for injection in vehicles such as saline, dextrose solution, serum albumin and Ringer's solution. The administration of such active ingredient may be by intravenous, intramuscular or subcutaneous route. Other routes of administration, which may establish the desired blood levels of the respective ingredients, are comprised by the present invention. For example, administration may be by various parenteral routes such as subcutaneous, intravenous, intradermal, intramuscular, intraperitoneal, intranasal, transdermal, oral, or buccal routes.

The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages. Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl oleate or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The optimal dose of active ingredient may be appropriately selected according to the route of administration, patient conditions and characteristics (sex, age, body weight, health, size), extent of symptoms, concurrent treatments, frequency of treatment and the effect desired. Adjustment and manipulation of established dosage ranges are well within the ability of those skilled.

Usually a daily dosage of active ingredient can be about 0.01 to 100 milligrams per kilogram of body weight. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

The present invention has been described with reference to the specific embodiments, but the content of the descriptions comprises all modifications and substitutions, which can be brought by a person skilled in the art without extending beyond the meaning and purpose of the claims.

The invention will now be described by means of the following Examples, which should not be construed as in any way limiting the present invention. The examples will refer to the Figures specified here below.

### EXAMPLES

### Example 1

### Cloning of Human RANTES by PCR

Human RANTES was cloned from a human bone marrow λGT11 cDNA library (Clontech) by PCR. Briefly, total cDNA inserts in the bone marrow library were first amplified using λGT11 primers which flanked the Eco R1 cloning site in a 100 μl reaction containing 2 μl of phage stock (10⁶ pfus), 10 mM Tris-HCl pH 8.3, 50 mM KCl, 1.5 mM MgCl₂, 0.2 mM dNTPs, 2.5 units Amplitaq™ (Perkin Elmer-Cetus) and 1 μl of each primer (λGT11 PCR-1 (forward primer) 5′ GATTGGTGGCGACGACTCCT and λGT11 PCR-2 (reverse primer) 5′ CAACTGGTAATGGTAGCGAC) for 30 cycles of 95 C 2 min, 55 C 2 min and 72 C 5 min in a Techne PHC-2 thermal cycler. One tenth of the reaction mixture was then subjected to a 2nd round of PCR in a 100 μl reaction now containing 1 μM each of specific primers (RANTES-1 5′ CCATGAAGGTCTCCGCGGCAC sense and RANTES-2 5′ CCTAGCTCATCTCCAAAGAG antisense) based on the published RANTES sequence (Schall T. J et al, 1988) for 30 cycles of 95 C 2 min, 55 C 2 min and 72 C 2 min. PCR products were visualized on 3% Nu-Sieve (FMC) agarose gels stained with 0.5 μg/ml ethidium bromide and bands migrating at the predicted size of RANTES cDNA (278 bp) were gel purified by standard methods (Sambrook J. et al., (1989). Gel purified DNA was then rendered blunt-ended by sequential treatment with T4 polynucleotide kinase (New England Biolabs) according to the manufacturers' instructions, in a total volume of 50 μl for 1 h at 37 C. After this time, 2.5 μl of 2.5 mM dNTPs and 1 μl of E. coli DNA polymerase I Klenow fragment (New England Biolabs) were added and the incubation continued for a further 30 min at 37 C. The reaction mixture was then heat inactivated at 70 C for 30 min and then extracted once with Tris-HCl pH 8.0 saturated phenol/chloroform (1:1 v/v). DNA was precipitated by addition of 10 μl 3M sodium acetate pH 5.5, 1 μl glycogen (20 mg/ml) (Boehringer) and 250 μl ethanol at −20 C. The DNA was recovered by centrifugation at 10 000×g for 20 min at 4 C and washed with 70% ethanol. The final pellet was resuspended in sterile water at a concentration of 10 ng/μl.

Blunt-ended PCR product (10 ng) was ligated to 50 ng of Eco RV digested, alkaline phosphatase treated pBluescript II SK-plasmid (Stratagene) in a 20 μl volume using 2 μl of T4 DNA ligase (400 000 units/ml) (New England Biolabs) for at least 16 h at 15 C. Ligation products were diluted to 100 μl with 1×TE (10 mM Tris-HCl pH 8.0/1 mM EDTA) and phenol/chloroform extracted as described previously. Ligation products were precipitated by the addition of 10 μl 3M sodium acetate pH 5.5, 1 μl glycogen (20 mg/ml) and 250 μl ethanol for 15 min at −70 C. DNA was recovered by centrifugation as described above and resuspended in 10 μl of sterile water. Five μl of resuspended ligation products were then electroporated into electrocompetent E. coli strain XL-1 blue (40 μl) using a Bio Rad Gene pulser according to the manufacturers' instructions. Following electroporation, 1 ml of LB medium was added and cells were grown at 37 C for 1 h. After this time, 100 μl aliquots of the culture medium were plated on LB plates containing 100 μg/ml of ampicillin and grown up for 16 h at 37 C. Individual bacterial colonies were then picked into 5 ml of LB medium containing 100 μg/ml of ampicillin and grown overnight at 37 C. Small scale plasmid DNA preparations (mini-preps) were then made from 3 ml of each culture using a Wizard™ mini-prep DNA purification system (Promega) according to the manufacturers' instructions. Three μl aliquots of mini-prep DNA was then digested with restriction enzymes Hind III and Eco RI (both from New England Biolabs) according to the manufacturers' instructions in a reaction volume of 15 μl. Reaction products were analysed on 1% agarose gels containing 0.5 μg/ml ethidium bromide. Mini-prep DNAs which yielded an insert size of approximately 280 bp were then subjected to DNA sequence analysis using T3 and T7 primers and Sequence (USB) according to the manufacturers' instructions.

The pBluescript II SK-cloning vector (pBSIISK⁻/RANTES) was prepared as follows: 20 μl of CsCl gradient purified plasmid was digested in a reaction volume of 100 μl for 2 h at 37 C with 200 units of Eco RV (New England Biolabs) according to the manufacturers' instructions. After 2 h, the digested vector was treated with 10 μl of calf intestinal alkaline phosphatase (20 units/ml) (Boehringer) for a further 30 min at 37 C. The reaction mixture was inactivated by heating at 68 C for 15 min and then extracted once with Tris-HCl pH 8.0 saturated phenol/chloroform (1:1 v/v). Plasmid DNA was precipitated by addition of 10 μl 3M sodium acetate pH 5.5 and 250 μl ethanol at −20 C. The DNA was recovered by centrifugation at 10 000×g for 20 min at 4 C, washed with 70% ethanol. The final pellet was resuspended in sterile water at a concentration of 50 ng/ml.

Sequencing revealed that all clones obtained were identical to the published sequence except for a single base change at nucleotide 22 in the PCR sequence (G22C) which would result in an Arg to Pro change in the proposed signal sequence of the RANTES propeptide.

TABLE 1
PCR Primers

| | |
|---|---|
| λGT11-PCR-1 FP | 5′ GATTGGTGGCGACGACTCCT 3′ |
| SEQ ID NO: 11 | |
| λGT11-PCR-2 RP | 5′ CAACTGGTAATGGTAGCGAC 3′ |
| SEQ ID NO: 12 | |
| RANTES-1 sense | 5′ CCATGAAGGTCTCCGCGGCAC 3′ |
| SEQ ID NO: 13 | |
| RANTES-2 antisense | 5′ CCTAGCTCATCTCCAAAGAG 3′ |
| SEQ ID NO: 14 | |

Mature Wildtype (wt) RANTES has the following amino acid sequence:

SPYSSDTTPCCFAYIARPLPRAHIKEYFYTSGKCSNPAVVFVTRKNRQVC
ANPEKKWVREYINSLEMS.

It is herein referred to as SEQ ID NO. 1

### Example 2

### Generation of RANTES (S1C)

The ORF of RANTES was contained in the plasmid DNA template, pBSIISK⁻/RANTES, as described in example 1. The point mutation S1C, was introduced by PCR amplification using RANTES (S1C) Nde I FP and RANTES (EcoR I) RP. The forward primer [RANTES (S1C) Nde I FP] was designed in such a way to introduce the desired mutation i.e. S1C. RANTES (S1C) was then cloned into 5′Nde/and 3′ EcoR I sites of pET20b(+) vector.

PCR Reaction:

pBSIISK⁻/RANTES was used as PCR template to generate RANTES (S1C) with 5′ Nde I and 3′ EcoR I sites. The PCR reaction, (in a final volume of 50 μl) contained respectively: 1 μl (25 ng) of plasmid pBSIISK⁻/RANTES, 4.0 μl dNTPs (10 mM stock), 5 μl of 10× Pwo polymerase buffer, 1.0 μl each of gene specific primer (to give a final concentration of 80 pico-moles) (RANTES (S1C) Nde I FP and RANTES (EcoR I) RP), and 0.5 μl (5 Units/μl) Pwo DNA polymerase (Roche). The PCR reaction was performed using an initial denaturing step of 95° C. for 5 min, followed by 30 cycles of 94° C. for 30 s; 57° C. for 30 s and 72° C. for 30 sec; and a final extension cycle of 72° C. for 5 minutes and a holding cycle of 4° C. An aliquot of amplification product was visualized on 1.6% agarose gel in 1×TAE buffer in order to verify that the product was of the expected molecular weight (231 bp). The PCR amplified product was purified using the QIAquick Gel Extraction Kit (Qiagen). A 1 μl aliquot was visualized on 1.6% agarose gel.

Restriction Digestion:

The Gel purified RANTES (SiC) product was digested with Nde I and EcoR I enzymes. The Restriction digestion reaction (in a final volume of 100 μl) contained: 45 μl (2 μl) of the Gel purified DNA, 10 μl of 10×NEB buffer 2, 1 μl (10 U) each of Nde I and EcoR I enzyme. The digestion was carried out for 1 hr at 37° C. The digested product was directly gel purified using the QIAquick Gel Extraction Kit (Qiagen). A 1 μl aliquot was visualized on 1.6% agarose gel.

Vector Preparation

pET20b(+) vector was digested with Nde I and EcoR I enzymes. The Restriction digestion reaction (in a final volume of 250 μl) contained: 20 μl (5 μl) of the vector DNA, 25 μl of 10×NEB buffer 2, 1 μl (10 U) each of Nde I and EcoR I enzyme. The digestion was carried out for 1 hr at 37° C. The digested product was directly gel purified using the QIAquick Gel Extraction Kit (Qiagen). A 1 μl aliquot was visualized on 1.6% agarose gel.

Dephosphorylation:

The digested vector was dephosphorylated using Shrimp alkaline Phosphatase (SAP). The reaction (in a final volume of 30 μl) contained: 18 μl (900 ng) of digested vector DNA, 3 μl of 10×SAP buffer, and 3 μl (1 U/μl) of SAP enzyme. The reaction mix was incubated at 37° C. for 1 hr and the enzyme was heat inactivated by incubating at 65° C. for 20 min.

Ligation of pET20b (+) and RANTES (S1C):

The ligation was carried out in the ratio 1:10: The ligation reaction (in a final volume of 20 μl) contained: 2 μl (25 ng) of RANTES (S1C), 1 μl (30 ng) of pET20b (+) each of 2 μl of 10×NEB T4 DNA ligase buffer, 1 μl of (1:4) NEB Ligase enzyme. The ligation mix was incubated at 16° C. overnight.

Transformation:

The ligation mix was used to transform DH5α strain as follows: a 50 μl aliquot of DH5α cells was thawed on ice and 10 μl of ligation mixture was added. The mixture was incubated for 30 min on ice and then heat shocked by incubation at 42° C. for exactly 2 min. Samples were returned to ice and 300 μl of warm SOC media (room temperature) was added. Samples were incubated with shaking (250 rpm) for 1 h at 37° C. The transformation mixture was then plated on Luria agar plates containing Ampicillin (100 μl/ml) and incubated overnight at 37° C.

Screening

Ten numbers of transformants were picked and patched on LB agar plates containing Ampicillin (100 μl/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of 10×Taq polymerase buffer, 0.5 μl of screening primers (to give a final concentration of 100 picomoles) (T7P and RANTES (EcoRI) RP) and 0.5 μl of Taq DNA polymerase.

The conditions for the screening PCR reaction were: 94° C. for 2 min, followed by 30 cycles of 94° C. for 30 s; 57° C. for 30 s and 72° C. for 45 sec; and a final extension cycle of 72° C. for 5 minutes and a holding cycle of 4° C. The PCR products were loaded onto a 1.6% agarose gel to verify the fragment size.

Plasmid DNA Preparation and Sequencing:

One positive clone was selected and plasmid mini-prep DNA was prepared from 5 ml cultures using QIAprep Spin Miniprep kit (Qiagen). Plasmid DNA (150-200 ng) was subjected to DNA sequencing with T7P and T7T primers using the CEQ Dye Terminator Cycle sequencing Quick Start Kit (Beckman Coulter P/N 608120) according to the manufacturer's instructions. The primer sequences are shown in Table 2. Sequencing reactions were analyzed on CEQ 2000 XL DNA analysis system (Beckman Coulter P/N 608450).

Sequence confirmed clone was designated as pET20b (+)/RANTES (S1C).

TABLE 2
PCR primers

| | |
|---|---|
| RANTES (S1C) Nde I FP | 5′TCC GCA AAA CAT ATG TGC |
| SEQ ID NO:2 | CCA TAT TCC TCG GAC ACC 3′ |
| RANTES EcoRi RP | 5′CAA GAA TTC TCA GCT CAT |
| SEQ ID NO:3 | CTC CAA AGA GTT GAT 3′ |
| T7P SEQ ID NO: 4 | 5′TAA TAC GAC TCA CTA TAG |
| | GG 3′ |
| T7T SEQ ID NO: 5 | 5′GCT AGT TAT TGC TCA GCG |
| | G 3′ |

S1C RANTES has the following sequence:

CPYSSDTTPCCFAYIARPLPRAHIKEYFYTSGKCSNPAVVFVTRKNRQVC
ANPEKKWVREYINSLEMS.

It is herein referred to as SEQ ID NO:6.

### Example 3

### Generation of RANTES (S4C)

The ORF of RANTES was contained in the plasmid DNA template, pBSIISK⁻/RANTES, as described in example 1. The point mutation S4C, was introduced by PCR amplification using RANTES (S4C) Nde I FP and RANTES (EcoR I) RP. The Forward primer [RANTES (S4C) Nde I FP] was designed in such a way to introduce the desired mutation i.e. S4C. RANTES (S4C) was then cloned into 5′ Nde I and 3′ EcoR I sites of pET20b(+) vector.

PCR Reaction:

pBSIISK⁻/RANTES was used as PCR template to generate RANTES (S4C) with 5′ Nde I and 3′ EcoR I sites. The PCR reaction, (in a final volume of 50 μl) contained respectively: 1 μl (25 ng) of plasmid pBSIISK⁻/RANTES, 4.0 μl dNTPs (10 mM stock), 5 μl of 10×Pwo polymerase buffer, 1.0 μl each of gene specific primer (to give a final concentration of 80 pico-moles) (RANTES (S4C) Nde I FP and RANTES (EcoR I) RP), and 0.5 μl Pwo DNA polymerase [5 U/μl] (Roche). The PCR reaction was performed using an initial denaturing step of 95° C. for 5 min, followed by 30 cycles of 94° C. for 30 sec; 57° C. for 30 sec and 72° C. for 30 sec; and a final extension cycle of 72° C. for 5 minutes and a holding cycle of 4° C. An aliquot of amplification product was visualized on 1.6% agarose gel in 1×TAE buffer in order to verify that the product was of the expected molecular weight (231 bp). The PCR amplified product was purified using the QIAquick Gel Extraction Kit (Qiagen). A 1 μl aliquot was visualized on 1.6% agarose gel.

Restriction Digestion:

The Gel purified RANTES (S4C) product was digested with Nde I and EcoR I enzymes. The Restriction digestion reaction (in a final volume of 100 μl) contained: 45 μl (2 μl) of the Gel purified DNA, 10 μl of 10×NEB buffer 2, 1 μl (10 U) each of Nde I and EcoR I enzyme. The digestion was carried out for 1 hr at 37° C. The digested product was directly gel purified using the QIAquick Gel Extraction Kit (Qiagen). A 1 μl aliquot was visualized on 1.6% agarose gel.

Vector Preparation

pET20b(+) vector was digested with Nde I and EcoR I enzymes. The Restriction digestion reaction (in a final volume of 250 μl) contained: 20 μl (5 μl) of the vector DNA, 25 μl of 10×NEB buffer 2, 1 μl (10 U) each of Nde I and EcoR I enzyme. The digestion was carried out for 1 hr at 37° C. The digested product was directly gel purified using the QIAquick Gel Extraction Kit (Qiagen). A 1 μl aliquot was visualized on 1.6% agarose gel.

Dephosphorylation:

The digested vector was dephosphorylated using Shrimp alkaline Phosphatase (SAP). The reaction (in a final volume of 30 μl) contained: 18 μl (900 ng) of digested vector DNA, 3 μl of 10×SAP buffer, and 3 μl (1 U/μl) of SAP enzyme. The reaction mix was incubated at 37° C. for 1 hr and the enzyme was heat inactivated by incubating at 65° C. for 20 min.

Ligation of pET20b (+) and RANTES (S4C):

The ligation was carried out in the ratio 1:10: The ligation reaction (in a final volume of 20 μl) contained: 2 μl (25 ng) of RANTES (S4C), 1 μl (30 ng) of pET20b (+) each of 2 μl of 10×NEB T4 DNA ligase buffer, 1 μl of (1:4) NEB Ligase enzyme. The ligation mix was incubated at 16° C. overnight.

Transformation:

The ligation mix was used to transform DH5α strain as follows: a 50 μl aliquot of DH5α cells was thawed on ice and 10 μl of ligation mixture was added. The mixture was incubated for 30 min on ice and then heat shocked by incubation at 42° C. for exactly 2 min. Samples were returned to ice and 300 μl of warm SOC media (room temperature) was added. Samples were incubated with shaking (250 rpm) for 1 h at 37° C. The transformation mixture was then plated on Luria agar plates containing Ampicillin (100 μg/ml) and incubated overnight at 37° C.

Screening

Ten numbers of transformants were picked and patched on LB agar plates containing Ampicillin (100 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of 10×Taq polymerase buffer, 0.5 μl of screening primers (to give a final concentration of 100 picomoles) [T7P and RANTES (EcoR I) RP] and 0.5 μl of Taq DNA polymerase.

The conditions for the screening PCR reaction were: 94° C. for 2 min, followed by 30 cycles of 94° C. for 30 s; 57° C. for 30 s and 72° C. for 45 sec; and a final extension cycle of 72° C. for 5 minutes and a holding cycle of 4° C. The PCR products were loaded onto a 1.6% agarose gel to verify the fragment size.

Plasmid DNA Preparation and Sequencing:

One positive clone was selected and plasmid mini-prep DNA was prepared from 5 ml cultures using QIAprep Spin Miniprep kit (Qiagen). Plasmid DNA (150-200 ng) was subjected to DNA sequencing with T7P and T7T primers using the CEQ Dye Terminator Cycle sequencing Quick Start Kit (Beckman Coulter P/N 608120) according to the manufacturer's instructions. The primer sequences are shown in Table 3. Sequencing reactions were analyzed on CEQ 2000 XL DNA analysis system (Beckman Coulter P/N 608450).

Sequence confirmed clone was designated as pET20b (+)/RANTES (S4C).

TABLE 3
PCR primers

| | |
|---|---|
| RANTES (S4C) Nde I FP | 5′ TCC GCA AAA CAT ATG TCC |
| SEQ ID NO:7 | CCA TAT TGC TCG GAC ACC 3′ |
| RANTES EcoRi RP | 5′ CAA GAA TTC TCA GCT CAT |
| SEQ ID NO:3 | CTC CAA AGA GTT GAT 3′ |
| T7P SEQ ID NO: 4 | 5′ TAA TAC GAC TCA CTA TAG |
| | GG 3′ |
| T7T SEQ ID NO: 5 | 5′ GCT AGT TAT TGC TCA GCG |
| | G 3′ |

S4C RANTES has the following sequence:

SPYCSDTTPCCFAYIARPLPRAHIKEYFYTSGKCSNPAWFVTRKNRQVQA
NPEKKWVREYINSLEMS.

It is herein referred to as SEQ ID NO:8.

### Example 4

### Generation of RANTES (S5C)

The ORF of RANTES was contained in the plasmid DNA template, pBSIISK⁻/RANTES, as described in example 1. The point mutation S5C, was introduced by PCR amplification using RANTES (S5C) Nde I FP and RANTES (EcoR I) RP. The Forward primer [RANTES (S5C) Nde I FP] was designed in such a way to introduce the desired mutation i.e. S5C. RANTES (S5C) was then cloned into 5′ Nde I and 3′ EcoR I sites of pET20b(+) vector.

PCR Reaction:

pBSIISK⁻/RANTES (SPRI template) was used as PCR template to generate RANTES (S5C) with 5′ Nde I and 3′ EcoR I sites. The PCR reaction, (in a final volume of 50 μl) contains respectively: 1 μl (25 ng) of plasmid pBSIISK⁻/RANTES, 4.0 μl dNTPs (10 mM stock), 5 μl of 10×Pwo polymerase buffer, 1.0 μl each of gene specific primer (to give a final concentration of 80 pico-moles) (RANTES (S5C) Nde I FP and RANTES (EcoR I) RP), and 0.5 μl [5 U/μl] Pwo DNA polymerase (Roche). The PCR reaction was performed using an initial denaturing step of 95° C. for 5 min, followed by 30 cycles of 94° C. for 30 s; 57° C. for 30 and 72° C. for 30 sec; and a final extension cycle of 72° C. for 5 minutes and a holding cycle of 4° C. An aliquot of amplification product was visualized on 1.6% agarose gel in 1×TAE buffer in order to verify that the product was of the expected molecular weight (231 bp). The PCR amplified product was purified using the QIAquick Gel Extraction Kit (Qiagen). A 1 μl aliquot was visualized on 1.6% agarose gel.

Restriction Digestion:

The Gel purified RANTES (S5C) product was digested with Nde I and EcoR I enzymes. The Restriction digestion reaction (in a final volume of 100 μl) contained: 45 μl (2 μl) of the Gel purified DNA, 10 μl of 10×NEB buffer 2, 1 μl (10 U) each of Nde I and EcoR I enzyme. The digestion was carried out for 1 hr at 37° C. The digested product was directly gel purified using the QIAquick Gel Extraction Kit (Qiagen). A 1 μl aliquot was visualized on 1.6% agarose gel.

Vector Preparation

pET20b(+) vector was digested with Nde I and EcoR I enzymes. The Restriction digestion reaction (in a final volume of 250 μl) contained: 20 μl (5 μl) of the vector DNA, 25 μl of 10×NEB buffer 2, 1 μl (10 U) each of Nde I and EcoR I enzyme. The digestion was carried out for 1 hr at 37° C. The digested product was directly gel purified using the QIAquick Gel Extraction Kit (Qiagen). A 1 μl aliquot was visualized on 1.6% agarose gel.

Dephosphorylation:

The digested vector was dephosphorylated using Shrimp Alkaline Phosphatase (SAP). The reaction (in a final volume of 30 μl) contained: 18 μl (900 ng) of digested vector DNA, 3 μl of 10×SAP buffer, and 3 μl (1 U/μl) of SAP enzyme. The reaction mix was incubated at 37° C. for 1 hr and the enzyme was heat inactivated by incubating at 65° C. for 20 min.

Ligation of pET20b (+) and RANTES (S5C):

The ligation was carried out in the ratio 1:10: The ligation reaction (in a final volume of 20 μl) contained: 2 μl (25 ng) of RANTES (S5C), 1 μl (30 ng) of pET20b (+) each of 2 μl of 10×NEB T4 DNA ligase buffer, 1 μl of (1:4) NEB Ligase enzyme. The ligation mix was incubated at 16° C. overnight.

Transformation:

The ligation mix was used to transform DH5α strain as follows: a 50 μl aliquot of DH5α cells was thawed on ice and 10 μl of ligation mixture was added. The mixture was incubated for 30 min on ice and then heat shocked by incubation at 42° C. for exactly 2 min. Samples were returned to ice and 300 μl of warm SOC media (room temperature) was added. Samples were incubated with shaking (250 rpm) for 1 h at 37° C. The transformation mixture was then plated on Luria agar plates containing Ampicillin (100 μg/ml) and incubated overnight at 37° C.

Screening

Ten numbers of transformants were picked and patched on LB agar plates containing Ampicillin (100 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of 10×Taq polymerase buffer, 0.5 μl of screening primers (to give a final concentration of 100 picomoles) (T7P and RANTES (EcoRI) RP) and 0.5 μl of Taq DNA polymerase.

The conditions for the screening PCR reaction were: 94° C. for 2 min, followed by 30 cycles of 94° C. for 30 s; 57° C. for 30 s and 72° C. for 45 sec; and a final extension cycle of 72° C. for 5 minutes and a holding cycle of 4° C. The PCR products were loaded onto a 1.6% agarose gel to verify the fragment size.

Plasmid DNA Preparation and Sequencing:

One positive clone was selected and plasmid mini-prep DNA was prepared from 5 ml cultures using QIAprep Spin Miniprep kit (Qiagen). Plasmid DNA (150-200 ng) was subjected to DNA sequencing with T7P and T7T primers using the CEQ Dye Terminator Cycle sequencing Quick Start Kit (Beckman Coulter P/N 608120) according to the manufacturer's instructions. The primer sequences are shown in Table 4. Sequencing reactions were analyzed on CEQ 2000 XL DNA analysis system (Beckman Coulter P/N 608450).

Sequence confirmed clone was designated as pET20b (+)/RANTES (S5C).

TABLE 4
PCR primers

| | |
|---|---|
| RANTES (S5C) Nde I FP | 5′ TCC GCA AAA CAT ATG TCC |
| SEQ ID NO:9 | CCA TAT TGC TCG GAC ACC 3′ |
| RANTES EcoRi RP | 5′ CAA GAA TTC TCA GCT CAT |
| SEQ ID NO:3 | CTC CAA AGA GTT GAT 3′ |
| T7P SEQ ID NO: 4 | 5′ TAA TAC GAC TCA CTA TAG |
| | GG 3′ |
| T7T SEQ ID NO: 5 | 5′ GCT AGT TAT TGC TCA GCG |
| | G 3′ |

S5C RANTES has the following sequence:

SPYSCDTTPCCFAYIARPLPRAHIKEYFYTSGKCSNPAVVFVTRKNRQVC
ANPEKKWVREYINSLEMS.

It is herein referred to as SEQ ID NO:10.

### Example 5

### Purification of Chemokine Variants

WT RANTES was expressed with a leader sequence MKKKWPR at the amino terminus and was purified as described in Proudfoot and Borlat, 2000, whereas the Ser-Cys mutants were expressed without this sequence.

The E. coli cell pellets were suspended in 3 volumes per g (wet weight) of cells (mg/ml) with cell breakage buffer: 50 mM Tris-HCl pH 8.0 containing 10 mM MgCl₂, 5 mM Benzamidine/HCl, 1 mM DTT, 1 mM PMSF, 20 mg/l DNAse and polytroned. The cells were broken by two passages on the French Pressure cell and the solution was centrifuged 30 min at 13′000 rpm (27,500×g). The inclusion bodies from the pellet were solubilised with 100 ml/g of cells (wet weight) in extraction buffer: 0.1 M Tris-HCl pH 8.5 containing 6 M guanidine-HCl and 1 mM DTT, polytroned, heated for 1 h at 60° C., cooled to room temperature and filtered with a 22 μm filter. The amount of protein was calculated by a calorimetric assay and applied to a Reverse Phase Source 30 Chromatography (RPC) column previously equilibrated in 0.1 M Tris-HCl pH 7.5. After loading, the RPC column was washed with 5 column volumes (CV) of 0.1 M Tris-HCl pH 7.5, then with 5 CV of 0.1% TFA and eluted with a 0-90% gradient of 90% acetonitrile in 0.1% TFA over 7 CV. The fractions were analysed by SDS-PAGE using NuPAGE 10% Bis-Tris gels run in MES buffer and pooled according to their chemokine quantity and purity.

The chemokine variants were renatured by infinite dilution. The pooled fractions from the RPC were adjusted to pH 8.0 with NaOH and added dropwise at 0.1 ml/min into the renaturation buffer: 0.1 M Tris/HCl pH8, containing 0.1 mM reduced glutathione, 0.01 mM oxidized glutathione at 4° C., to obtain a final protein concentration of 50 μg/ml. The renaturation solution was left overnight at 4° C. under agitation. The solution was filtered with a 22 μm filter, quantified, adjusted to pH 6.8 with HCl, diluted until the conductivity was below 10 mS in order to concentrate the renaturated chemokines by cation exchange chromatography. The column was freshly packed with SP Sepharose HP gel (Amersham®) and equilibrated with 50 mM potassium phosphate pH 6.8. After loading, the column was washed with 50 mM potassium phosphate pH 6.8 and eluted with a linear 0-2 M NaCl gradient in the same buffer over 10 CV. Fractions were analysed by SDS-PAGE as described above, pooled according to their chemokine content and purity, and the pool was quantified by UV spectra.

### Example 6

### PEGylation of the Free-Cysteine Chemokine Variants (S1C, S4C, S5C)

Free cysteine variants having the amino acid sequences of SEQ ID NO:6, SEQ ID NO:8 and SEQ ID NO:10 were PEGylated directly following the cation exchange chromatography after concentration by adding 25 equivalents of mPEG-MAL 5 kDa (NEKTAR®) in 50 mM potassium phosphate pH 6.8 (MAL=maleimide). The reaction was incubated for 2 h at RT. The reaction was analyzed at various time points by SDS-PAGE (NuPAGE 10% Bis-Tris with MES buffer) and RP-HPLC on a C8 column. After 2 h of reaction the excess PEG was quenched with 1 M Tris/HCl pH 8. The excess PEG was removed by cation exchange chromatography using SP Sepharose HP equilibrated with 50 mM sodium acetate pH 4.5. The proteins were eluted with a linear 0-2 M NaCl gradient in the same buffer over 10 CV. The fractions were analyzed by SDS-NuPAGE 10% Bis-Tris with MES buffer. The PEGylated and the unpegylated proteins were then separated by HPLC and fractions analysed by SDS-NuPAGE 10% Bis-Tris with MES buffer.

The amino terminal methionine was removed with Methionine Amino Peptidase (MAP) (PeproTech) by digestion for 48 h at room temperature in 0.1 M Tris/HCl pH 8.0 using an enzyme:substrate ratio of 1:10,000 (w:w).

The free cysteines in the chemokine variants S1C, S4C, S5C were PEGylated via their side chains and used herein as controls compared to RANTES which was PEGylated on the amino terminal residue via the α-amino group.

### Example 7

### N-Terminal PEGylation

Mature WT RANTES was solubilized at 1 mg/ml in 50 mM potassium phosphate pH 6.0. Six equivalents of mPEG-ButyrALD 2 or 5 kDa (Nektar®) were added to the solution in the presence of 30 μM BH₃CNNa as catalyst (ALD=aldehyde). The reaction was left 20 h at room temperature. The reaction was followed at various time points by SDS-NuPAGE 10% Bis-Tris wih MES buffer. The N terminal PEGylated variants were then purified by cation exchange chromatography and RP-HPLC as described above. The PEGylation was done via the α-amino group of the amino terminal residue.

### Example 8

### Competition Equilibrium Receptor Binding Assay

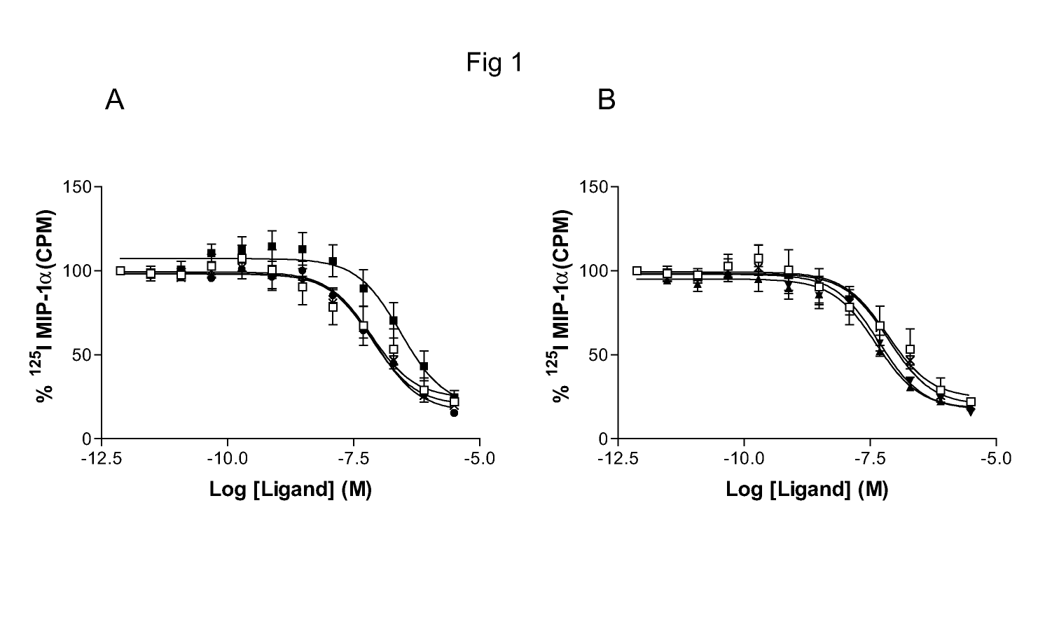

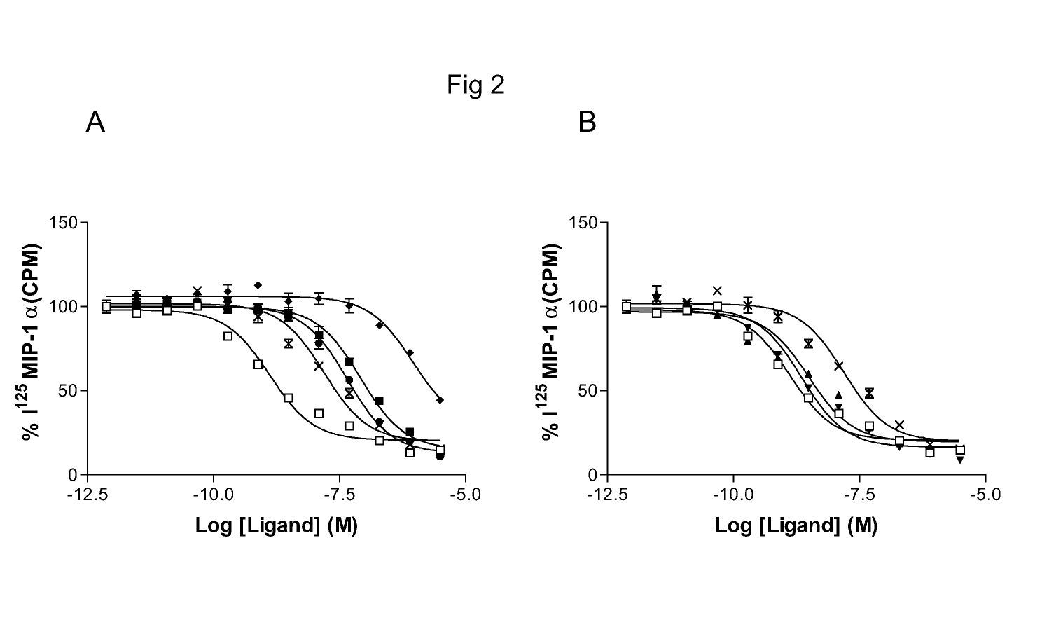

The assay was carried out on membranes isolated from CHO transfectants expressing CCR1 and CCR5 from Invitrogen® using a scintillation proximity assay (Alouani, 2000). The unlabelled chemokines were prepared in triplicate by serial dilution in the range of 10⁻⁶ to 10⁻¹² M in 50 mM HEPES pH 7.5, containing 1 mM CaCl₂, 5 mM MgCl₂ and 0.5% bovine serum albumin (binding buffer). Wheatgerm scintillation proximity assay beads (Amersham®) were solubilized at 50 mg/ml in PBS, diluted to 10 mg/ml in binding buffer and put at 250 μg/well. Membranes were diluted to 80 μg/ml in binding buffer and put at 2 μg/well in the assay plates. ¹²⁵I-MIP-1α (Amersham®) was put at 0.1 nM/well and finally unlabelled chemokine was added into the plate. The plates were incubated for 3 h at room temperature with agitation and radioactivity was read with a Wallac counter. See FIGS. 1 and 2.

### Example 9

### In vitro chemotaxis assay

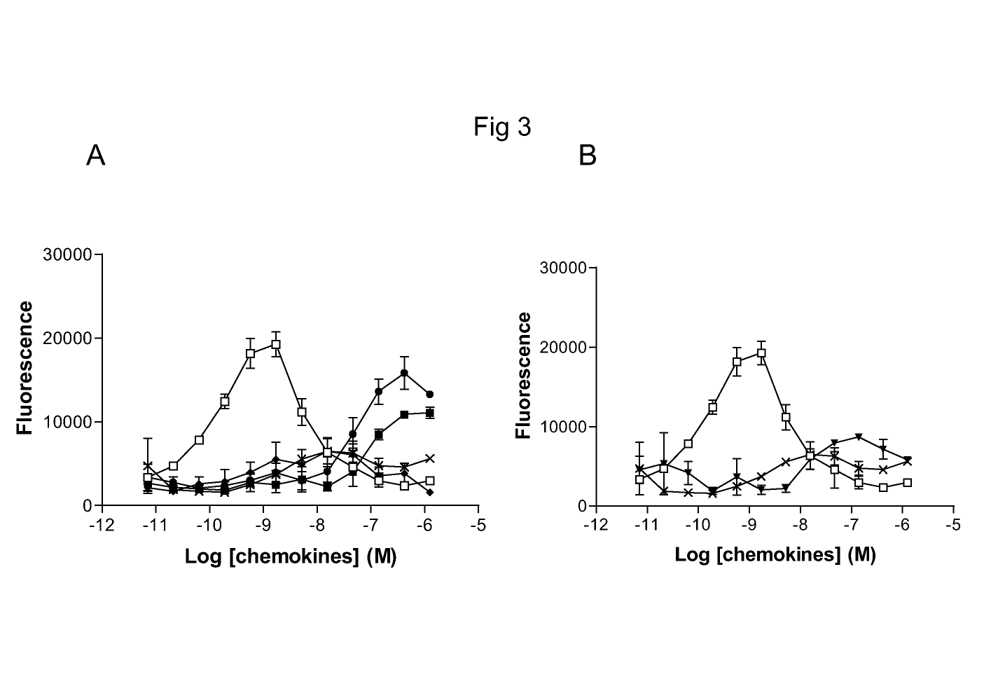

The chemotaxis assays were performed with L1.2/CCR5 transfectant cells. Cells were cultured in RPMI 1640 medium containing 1% penicillin, 1% L-glutamine, 10% FES and 1 μM de β-mercaptoethanol until a concentration of approximately 1×10⁶/ml. The transfectant cells were activated overnight with 5 mM butyric acid prior to the day of the chemotaxis assay. After washing with PBS, the cells were washed and incubated in the chemotaxis medium (RPMI 1640 without red phenol containing heat inactivated 5% FCS) at a concentration of 1×10⁶/ml. Chemotaxis was assayed in ChemoTx 96 well plate (Neuro Probe Inc.®) with 5 μM pores. The chemokines were placed in triplicate in the lower wells with appropriate dilutions in chemotaxis medium covering the range 10⁻¹²-10⁻⁶ M and covered with the membrane. The cells were placed on the upper surface of the membrane at 2.10⁴ cells/well and the plates were incubated 2 h at 37° C. with x % CO₂. The membrane was washed with PBS and the bottom wells were transferred into well flat bottom black plates (Costar®) using funnel adapters (Neuro Probe Inc.®). The black plates were frozen for a minimum of 1 h at −80° C., thawed and the number of migrated cells was measured using the CyQuant cell proliferation assay kit (Molecular Probes®). See FIG. 3.

### Example 10

### Inhibition of in Vitro Chemotaxis Assay

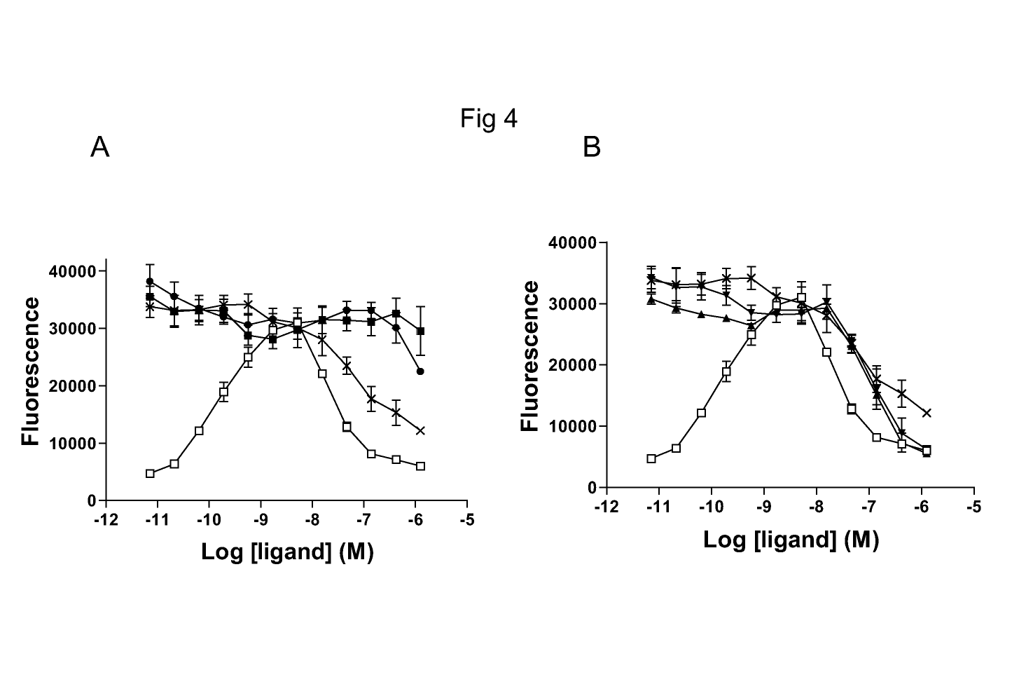

The chemotaxis assay was carried out as described above with the following modification. RANTES was incubated at a constant concentration of 1 nM with increasing concentrations of the inhibitors in the range of 10⁻¹²-10⁻⁶ M. see FIG. 4.

### Example 11

### Inhibition of Irritant Induced Contact Hypersensitivity In Vivo

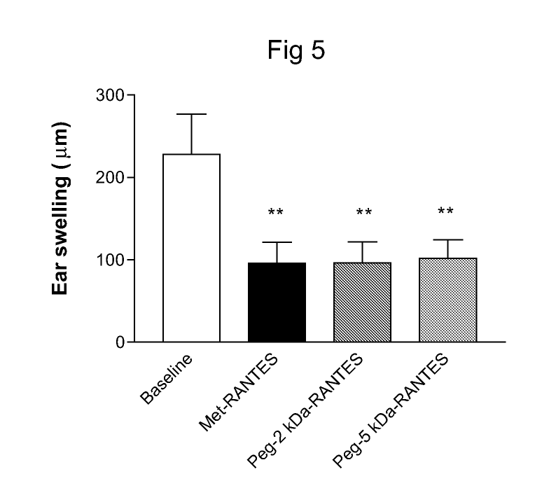

The ICD model is based on a chemical irritant, croton oil, a mixture of different phorbol esters. A 2% solution of croton oil in acetone/olive oil (5:1) is applied to both surfaces of the right ear. Balb/c mice were irritated with 2% croton oil in acetone/olive oil at time 0 and treated after 30′ with RANTES antagonist, either Met-RANTES or Peg-2 kDa-RANTES or Peg-5 kDa-RANTES at 0.5 mg/kg via i.p. injection. Dexamethasone at 0.5 mg/kg via s.c. was used as control. The ear swelling is followed by measuring the ear thickness of both ears at 3 time points and calculating the mean per ear, and the area under the curve (AUC) calculated. At 6 h after challenge mice are sacrificed and an ear-punch/ear is taken for further investigations. See FIG. 5.

Results

The mono-PEGylated RANTES proteins were easily separated from multi-PEGylated forms as well as the unpegylated protein. Their ability to bind to RANTES receptors was assessed on CCR1 and CCR5. All the PEGylated variants retained binding capacity to both receptors. However the variants which were PEGylated on the S1C, S4C and S5C mutants had reduced binding capacity for both receptors, whereas the amino terminal PEGylated RANTES retained affinity comparable to WT RANTES and Met-RANTES. See FIGS. 1 and 2. The IC₅₀ values are shown in Table 5.

TABLE 5
IC₅₀ values (nM) for CCR1 and CCR5 receptor binding

| Variant | CCR1 | CCR5 |
|---|---|---|
| WT RANTES | 23.3 ± 10 | 1.6 ± 0.9 |
| Met-RANTES | 73 ± 15 | 15.2 ± 2.8 |
| PEG-2 kDa-RANTES | 43 ± 16 | 10.6 ± 5.0 |
| PEG-5 kDa-RANTES | 50 ± 19 | 2.8 ± 2.0 |
| S1C-5 kDa-RANTES | 167 ± 27 | 41 ± 20 |
| S4C-5 kDa-RANTES | 84 ± 18 | 40 ± 10 |
| S5C-5 kDa-RANTES | — | 902 ± 50 |

Their ability to activate receptors was assessed by their activity in the induction of chemotaxis of L1.2/CCR5 transfectants. All of the PEGylated variants showed impaired activity in this assay (FIG. 3). In accordance with previous observations that modifications of the amino terminus of RANTES produce partial agonists on over-expressing recombinant cell lines (Proudfoot et al 1999) S1C-5 kDaPeg-RANTES and S4C-5 kDaPeg-RANTES showed partial agonist activity. S5C-5 kDaPeg-RANTES showed no agonist activity. Their ability to inhibit chemotaxis induced by 1 nM RANTES showed that both Peg-2 kDa-RANTES and Peg-5 kDa-RANTES were equipotent compared to Met-RANTES, whilst S1C-5 kDaPeg-RANTES and S4C-5 kDaPeg-RANTES had no inhibitory activity (FIG. 4). S5C-5 kDaPeg-RANTES was not tested in the inhibition assay in view of the fact that it had almost no affinity for CCR5 as ascertained in the receptor binding assay.

Since Met-RANTES has been shown to have important anti-inflammatory properties in vivo in several disease models, and while retaining receptor binding activity, has abrogated receptor activation, we propose that N-terminally PEGylated CC-chemokines, wherein the poly(ethyleneglycol) is attached via the α-amino-group of the amino-terminal residue, will similarly have anti-inflammatory activities. As shown in FIG. 5, effectively Met-RANTES, Peg-2 kDa-RANTES and Peg-5 kDa-RANTES were all able to inhibit the ear swelling observed in the murine Irritant Contact Dermatitis model by more than 50%.

### REFERENCES

Alouani, S., Methods Mol. Biol. 2000, 138: 135-141
Appay V and Rowland-Jones S L, Trends Immunol., 2001, 22:83-87
Brown et al., J. Pept. Sci., 2:40-46, 1996
Cleland J L et al., Curr. Opin. Biotechnology, 2001, 12:212-219
Edgerton M D et al., Methods Mol. Biol., 138:33-40, 2000
Gong J and Clark-Lewis I, J. Exp. Med., 1995, 181:631-640
Gong J H et al., J. Biol. Chem., 1996, 271:10521-7
Luo B and Prestwich G D, exp. Opin. Ther. Patents, 2001, 11:1395-1410
Proudfoot A et al., Immunol. Rev. 2000, 177:246-256
Proudfoot A and Borlat F., Methods Mol. Biol., 2000, 138:75-87
Proudfoot A et al., J. Biol Chem 1999 274 32478-32485
Sambrook J. Fritsch E. and Maniatis T., 1989, in Molecular Cloning: A Laboratory Manual. Cold Spring Harbor Laboratory Press, Cold Spring Harbor, N.Y., USA.
Schall T. J. et al., 1988 J. Immunol. 141, 1018-1025.
Vita C et al., J. Immunol. Methods, 266:53-65, 2002

1-16. (canceled)

17. An amino-terminally PEGylated CC-chemokine wherein the poly(ethyleneglycol) is attached to the amino-terminal residue via the α-amino group.

18. The amino-terminally PEGylated CC-chemokine according to claim 17, wherein the CC-chemokine is selected from RANTES (CCL5) or MCP-1 (CCL-2).

19. The amino-terminally PEGylated CC-chemokine according to claim 17, wherein the CC-chemokine is human wildtype RANTES, having the amino acid sequence of SEQ ID NO: 1.

20. The amino-terminally PEGylated CC-chemokine according to claim 17, wherein the poly(ethyleneglycol) has a molecular weight between 2-40 kDa.

21. The amino-terminally PEGylated CC-chemokine according to claim 20, wherein the poly(ethyleneglycol) is linear.

22. The amino-terminally PEGylated CC-chemokine according to claim 17, wherein said CC-chemokine has reduced receptor activation ability compared to the unpegylated wildtype chemokine.

23. The amino-terminally PEGylated CC-chemokine according to claim 17, wherein said CC-chemokine is a chemotaxis inhibitor.

24. The amino-terminally PEGylated CC-chemokine according to claim 17, wherein said CC-chemokine antagonizes the activity of the unpegylated wildtype chemokine.

25. A process for the preparation of an amino-terminally PEGylated CC-chemokine according to claim 17, wherein said process comprises PEGylation of a CC-chemokine by an acylation reaction.

26. The process according to claim 25, wherein a CC-chemokine and mPEG-ButyrALD are reacted in the presence of a catalyst.

27. A pharmaceutical composition comprising an amino-terminally PEGylated CC-chemokine according to claim 17 together with a pharmaceutically acceptable carrier.

28. A method for the treatment of autoimmune and inflammatory diseases, cancer, bacterial or viral infections comprising the administration of an effective amount of an amino-terminally PEGylated CC-chemokine according to claim 17 to a subject.


SEQUENCE LISTING (14 sequences)

SEQ ID NO: 1 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 2 - DNA, 36 bp, Artificial sequence
  PCR primer
tccgcaaaacatatgtgcccatattcctcggacacc

SEQ ID NO: 3 - DNA, 33 bp, Artificial sequence
  PCR primer
caagaattctcagctcatctccaaagagttgat

SEQ ID NO: 4 - DNA, 20 bp, Artificial sequence
  PCR primer
taatacgactcactataggg

SEQ ID NO: 5 - DNA, 19 bp, Artificial sequence
  PCR primer
gctagttattgctcagcgg

SEQ ID NO: 6 - protein, 68 aa, Homo sapiens
CPYSSDTTPCCFAYIARPLPRAHIKEYFYTSGKCSNPAVVFVTRKNRQVCANPEKKWVREYINSLEMS

SEQ ID NO: 7 - DNA, 36 bp, Artificial sequence
  PCR primer
tccgcaaaacatatgtccccatattgctcggacacc

SEQ ID NO: 8 - protein, 68 aa, Homo sapiens
SPYCSDTTPCCFAYIARPLPRAHIKEYFYTSGKCSNPAVVFVTRKNRQVCANPEKKWVREYINSLEMS

SEQ ID NO: 9 - DNA, 36 bp, Artificial sequence
  PCR primer
tccgcaaaacatatgtccccatattcctgcgacacc

SEQ ID NO: 10 - protein, 68 aa, Homo sapiens
SPYSCDTTPCCFAYIARPLPRAHIKEYFYTSGKCSNPAVVFVTRKNRQVCANPEKKWVREYINSLEMS

SEQ ID NO: 11 - DNA, 20 bp, Artificial sequence
  PCR primer
gattggtggcgacgactcct

SEQ ID NO: 12 - DNA, 20 bp, Artificial sequence
  PCR primer
caactggtaatggtagcgac

SEQ ID NO: 13 - DNA, 21 bp, Artificial sequence
  PCR primer
ccatgaaggtctccgcggcac

SEQ ID NO: 14 - DNA, 20 bp, Artificial sequence
  PCR primer
cctagctcatctccaaagag